(19) United States
(12) Patent Application Publication
(10) Pub. No.: US 2010/0241068 A1
(43) Pub. Date: Sep. 23, 2010

(54) Vascular cannula assembly with an improved structure for confining blood flow

(76) Inventor: Wei-Hui Chen, Taichung (TW)

Correspondence Address: COOPER & DUNHAM, LLP, 30 Rockefeller Plaza, 20th Floor, NEW YORK, NY, 10112, US

(21) Appl. No.: 12/383,225

(22) Filed: Mar. 20, 2009

(51) Int. Cl. A61M 39/22; A61M 25/10; A61M 1/00

(52) U.S. Cl. 604/96.01; 604/246; 604/151

(57) ABSTRACT

A vascular cannula assembly has a cannula, a tube dilator, a supporting helix and an inflatable cuff. The cannula has a distal end, a proximal end, a passage and a drainage segment. The drainage segment has multiple draining apertures communicating with the passage. A valve mounted on the distal end of the cannula can prevent air from entering the cannula. The tube dilator is selectively mounted in the passage and has a leading end and a handling end respectively protruding from the distal end and proximal end of the cannula. The supporting helix is mounted in the passage against the cannula. The inflatable cuff is mounted around the cannula between the distal end and the drainage segment. The side tubing is connected to the inflatable cuff. Since the inflatable cuff can be inflated to occlude blood draining from vena cava, use of the vascular cannula assembly can prevent injuries by snaring or looping of vessels during surgical procedures for cardiopulmonary bypass.

### BACKGROUND OF THE INVENTION

1. Field of the Invention

The present invention relates to vascular cannula used in draining venous blood during surgical procedures requiring cardiopulmonary bypass.

2. Description of the Prior Arts

The major risk factor for surgical operation lies in massive hemorrhage, especially for cardiac operations. Extracorporeal cardiopulmonary bypass is a major technique utilized to drain adequate volumes of blood from patients during cardiac procedures. In a routine cardiopulmonary bypass, two cannulae are respectively placed into the superior vena cava and the inferior vena cava for collecting venous blood returned to the patient's right atrium. The cannulae are secured with a snare around the vessels and ends of the vessels near the surgical site are further looped or sutured to prevent leakage of blood returned from vena cava or air entering the cannulae. However, looping and snaring of major blood vessels may cause their irreversible injuries and hence, the disastrous outcome of patients.

Accordingly, there is a need for a novel cannula assembly with an improved structure for resolving the aforesaid problems of the surgical procedures requiring cardiopulmonary bypass with conventional vascular cannulae.

### SUMMARY OF THE INVENTION

The main objective of the invention is to provide a vascular cannula assembly that has a structure for confining blood flow without causing undesired injuries to the blood vessels.

A vascular cannula comprises a cannula, a tube dilator, a supporting helix, an inflatable cuff and a side tubing.

The cannula is tubular and has a distal end, a proximal end, an inner surface, an outer surface, a valve and a drainage segment. The inner surface defines a passage. The passage is formed through the cannula from the distal end to the proximal end and forms an opening at the distal end. The valve is mounted over the opening at the distal end of the cannula. The drainage segment is formed adjacent to the distal end of the cannula and has multiple draining apertures. The draining apertures are formed through the drainage segment and communicate with the passage.

The tube dilator is selectively mounted in the passage of the cannula and has a leading end, a handling end and an attaching hole. The leading end is conical, selectively protrudes from the distal end and penetrates through the valve. The handling end protrudes from the proximal end of the cannula. The attaching hole is longitudinally formed through the tube dilator and mounted around a guide wire.

The supporting helix is located between the proximal end and the drainage segment of the cannula and is embedded in the cannula between the inner surface and the outer surface.

The inflatable cuff is mounted around the cannula between the distal end and the drainage segment.

The side tubing is connected to the inflatable cuff and has two ends. One of the two ends is connected to the inflatable cuff.

Based on the structure as the aforesaid, a vascular cannula assembly in accordance with the present invention can be inserted into inferior vena cava from surgical opening at the femoral vein. The tube dilator of each vascular cannula assembly is penetrated and led by a guide wire to a target site such as atriocaval junction as known in the field of the art. The tube dilator and the guidewire are withdrawn from the cannula through the valve. The valve close spontaneously to prevent air from entering the cannula causing air blockage once the right atrium is opened. The superior vena cava may be cannulated using the same principle or conventional method. The proximal end of the cannula of each vascular cannula assembly is then connected to an extracorporeal life supporting equipment, such as a heart-lung machine.

Consequently, blood draining from the inferior or superior vena cava flows through the draining apertures of the draining segment into the passage of the cannula of each vascular cannula assembly and finally converges in a venous reservoir of the extracorporeal life supporting equipment. Since the inflatable cuff is effective in occluding blood flow between vena cava and right atrium, when the vascular cannula assembly in accordance with the present invention is used in a surgical procedure for cardiopulmonary bypass, there is no needs for snaring or looping of vessels which contribute to irreversible injuries to vessels. Therefore, the vascular cannula assembly in accordance with the present invention has great advantages in use of surgeries.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a vascular cannula in accordance with the present invention with internal elements shown in broken lines;

FIG. 2 is an exploded perspective view of the vascular cannula in FIG. 1;

FIG. 3 is an enlarged perspective view of the vascular cannula in FIG. 1 with internal elements shown in phantom lines;

FIG. 4 is an enlarged side view of the vascular cannula in FIG. 1 with inflatable cuff shown inflated;

FIG. 5 is an operational side view of the vascular cannula in FIG. 1;

FIG. 6 is another operational side view of the vascular cannula in FIG. 1;

FIG. 7 is an operational scheme of the vascular cannula in FIG. 1 being used; and

FIG. 8 is another operational scheme of the vascular cannula in FIG. 1 being used when the tube dilator is withdrawn.

### DETAILED DESCRIPTION OF THE PREFERRED EMBODIMENTS

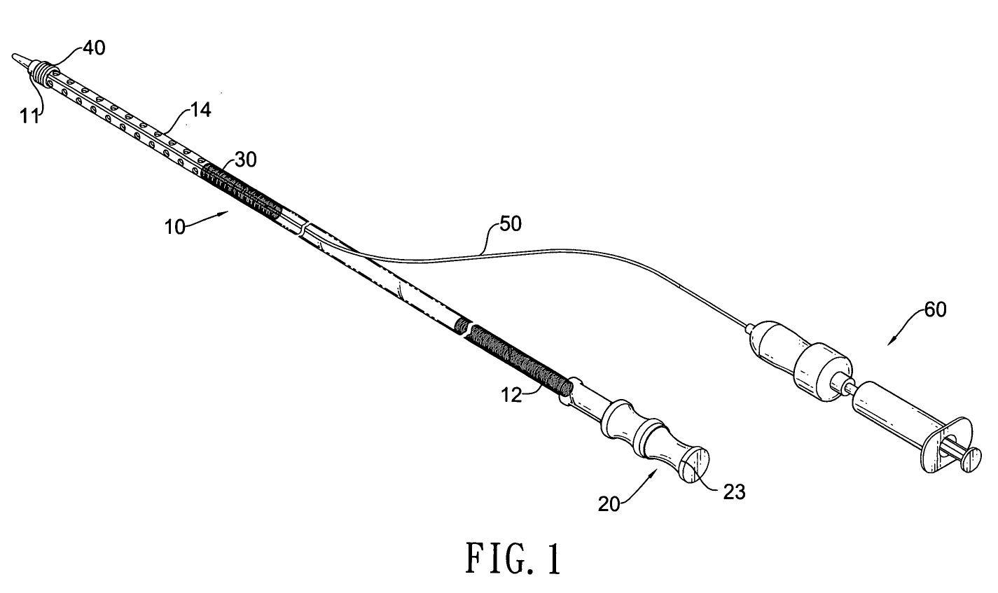

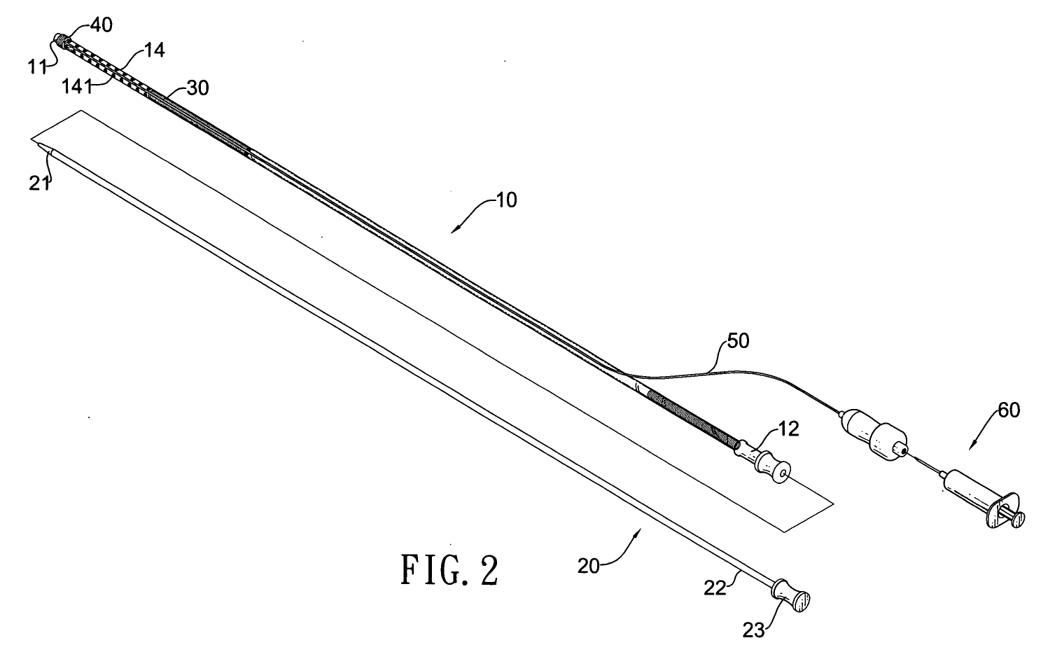

With reference to FIGS. 1 and 2, a vascular cannula assembly in accordance with the present invention comprises a cannula (10), a tube dilator (20), a supporting helix (30), an inflatable cuff (40), a side tubing (50) and an optional pumping mechanism (60).

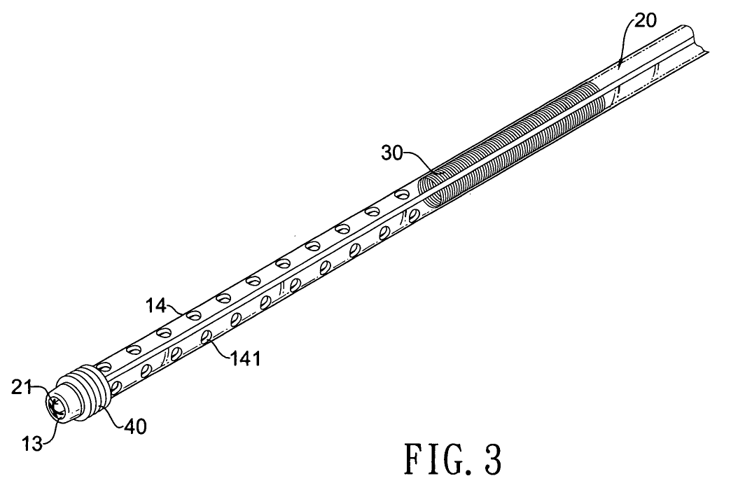

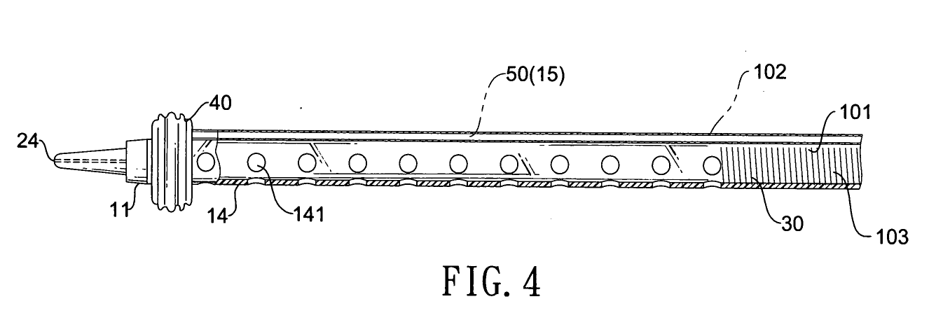

With further reference to FIGS. 3 and 4, the cannula (10) is tubular, has a distal end (11), a proximal end (12), an inner surface (101), an outer surface (102), a valve (13), a drainage segment (14) and an optional tunnel (15). The inner surface (101) defines a passage (103). The passage (103) is formed through the cannula (10) from the distal end (11) to the proximal end (12) and has an opening at the distal end (11) of the cannula (10). The valve (13) may be a resilient membrane having central stellate cleavage, is mounted over and selectively seals the opening at the distal end. The drainage segment (14) is formed adjacent to the distal end (11) of the cannula (10) and has multiple draining apertures (141). The draining apertures (141) are formed through the drainage segment (14) and communicate with the passage. The tunnel (15) extends along the cannula (10), preferably, is located in the drainage segment (14), between the inner surface (101) and the outer surface (102) from the distal end (11) to the proximal end (12).

The tube dilator (20) is cylindrical, is selectively mounted in the passage (103) of the cannula (10) and has a leading end (21), a handling end (22), an optional handle (23) and an attaching hole (24). The leading end (21) is conical, selectively protrudes from the distal end (11) and penetrates through the valve (13). The handling end (22) protrudes from the proximal end (12) of cannula (10). The handle (23) is mounted around the handling end (22) and may have a diameter greater than that of the passage (103) of the cannula (10). The attaching hole (24) is longitudinally formed through the tube dilator (20) and mounted around a guide wire. The attaching hole (24) has a desired diameter corresponding to a diameter of the guide wire, such that the tube dilator (20) is attached to the guide wire by friction and can be led by the guide wire to a target site as known in the field of the art.

The supporting helix (30) may be made of wire, is located between the proximal end (12) and the drainage segment (14) of the cannula (10) and is embedded in the cannula (10) between the inner surface (101) and the outer surface (102), preferably between the inner surface (101) and the tunnel (15).

The inflatable cuff (40) may be made of flexible materials, is mounted around the cannula (10) between the distal end (11) and the drainage segment (14).

The side tubing (50) is connected to the inflatable cuff (40), may be mounted in the tunnel (15) of the cannula (10) and has two ends. One end of the side tubing (50) is connected to the inflatable cuff (40).

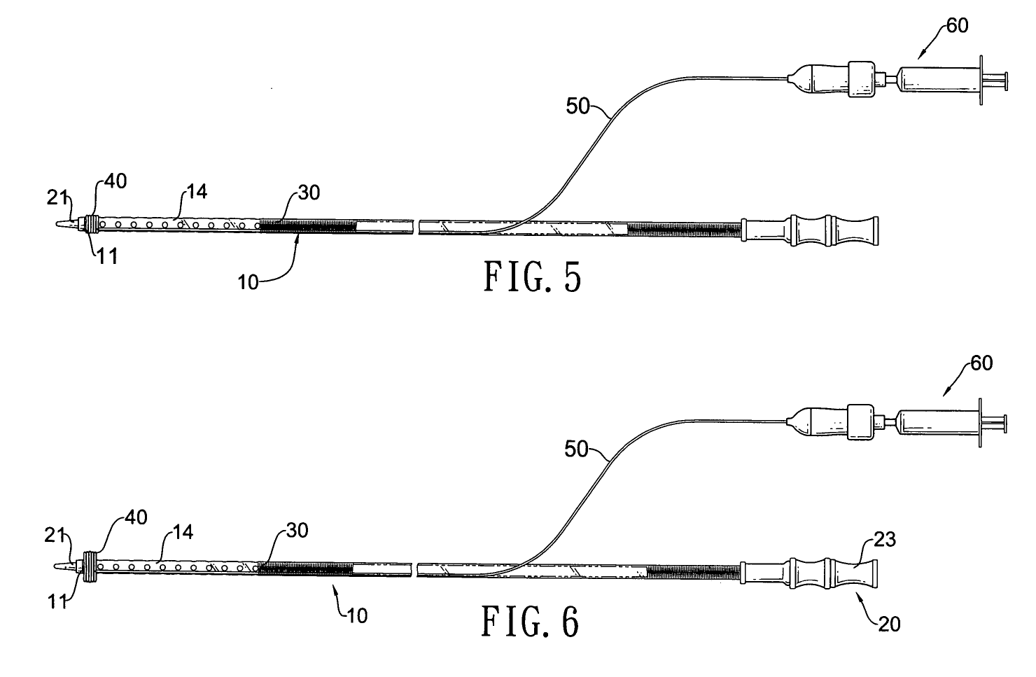

The pumping mechanism (60) is connected to the other end of the side tubing (50) and has a syringe and a check valve. The syringe is connected to the other end of the side tubing (50). The check valve is mounted between the side tubing (50) and the syringe. With reference to FIGS. 5 and 6, the inflatable cuff (40) can be inflated by the pumping mechanism (60).

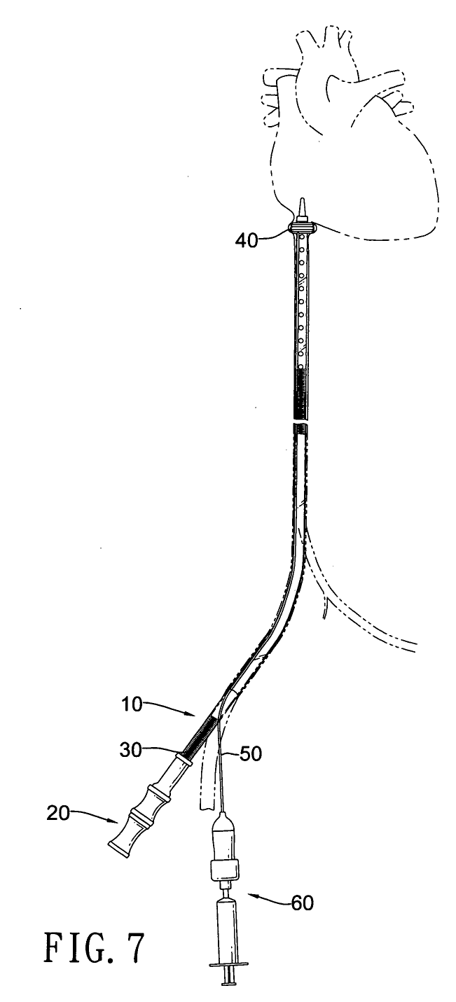

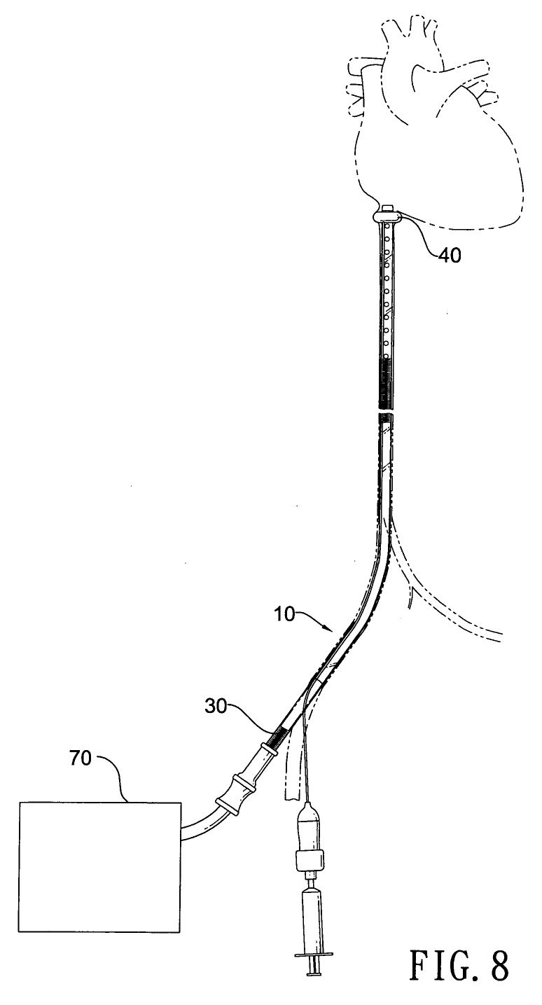

When used in venous drainage of cardiopulmonary bypass, one vascular cannula assembly in accordance with the present invention is inserted into inferior vena cava, and another may be inserted into superior vena cava in order to collect venous blood returned to the patient's right atrium. With reference to FIGS. 7 and 8, the tube dilator (20) of each vascular cannula assembly is led by a guide wire to atriocaval junction near the right atrium. The tube dilator (20) is withdrawn from the cannula (10) through the valve (13). The proximal end (12) of the cannula (10) of the vascular cannula assembly is connected to an extracorporeal life supporting equipment (70), such as a heart-lung machine. The inflatable cuff (40) is then inflated by the pumping mechanism (60) to prevent the blood from draining into the right atrium. Subsequently, the blood draining from the inferior or superior vena cava flows through the draining apertures (141) into the passage (103) of the cannula (10) of each vascular cannula assembly and finally converges in a venous reservoir of the extracorporeal life supporting equipment.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A vascular cannula assembly comprising
a cannula being tubular and having
a distal end;
a proximal end;
an inner surface defining a passage, the passage being formed through the cannula from the distal end to the proximal end and forming an opening at the distal end;
an outer surface;
a valve being mounted over and selectively sealing the opening at the distal end; and
a drainage segment being formed adjacent to the distal end of the cannula and having
multiple draining apertures being formed through the drainage segment and communicating with the passage;
a tube dilator being selectively mounted in the passage of the cannula and having
a leading end being conical, selectively protruding from the distal end and penetrating through the valve;
a handling end protruding from the proximal end of the cannula; and
an attaching hole being longitudinally formed through the tube dilator;
a supporting helix being located between the proximal end and the drainage segment of the cannula and being embedded in the cannula between the inner surface and the outer surface;
an inflatable cuff being mounted around the cannula between the distal end and the drainage segment; and
a side tubing being connected to the inflatable cuff and having
two ends, and one end being connected to the inflatable cuff.

2. The vascular cannula assembly of claim 1, wherein the valve is a resilient membrane having central stellate cleavage.

3. The vascular cannula assembly of claim 2 further comprising a pumping mechanism being connected to the other end of the side tubing.

4. The vascular cannula assembly of claim 3, wherein the pumping mechanism has a syringe being connected to the other end of the two ends of the side tubing.

5. The vascular cannula assembly of claim 3, wherein the cannula further comprises a tunnel extending along the cannula between the inner surface and the outer surface from distal end to the proximal end; the side tubing is mounted in the tunnel; and the supporting helix is between the inner surface and the tunnel.

6. The vascular cannula assembly of claim 4, wherein the cannula further comprises a tunnel extending along the cannula between the inner surface and the outer surface from distal end to the proximal end; the side tubing is mounted in the tunnel; and the supporting helix is between the inner surface and the tunnel.

7. The vascular cannula assembly of claim 5, wherein the tube dilator further comprises a handle being mounted around the handling end.

8. The vascular cannula assembly of claim 7, wherein the handle has a diameter greater than a diameter of the passage of the cannula.

9. The vascular cannula assembly of claim 4, wherein the pumping mechanism further comprises a check valve being mounted between the syringe and the side tubing.

10. The vascular cannula assembly of claim 6, wherein the pumping mechanism further comprises a check valve being mounted between the syringe and the side tubing.
